(19) United States
(12) Patent Application Publication
(10) Pub. No.: US 2007/0184556 A1
(43) Pub. Date: Aug. 9, 2007

(54) On-line monitoring method and device for a fossil fuel converter apparatus

(76) Inventor: Zhen Wang, Xi'an City (CN)

Correspondence Address: BERENATO, WHITE & STAVISH, LLC, 6550 ROCK SPRING DRIVE, SUITE 240, BETHESDA, MD, 20817, US

(21) Appl. No.: 10/590,929

(22) PCT Filed: Feb. 28, 2005
(86) PCT No.: PCT/CN05/00243

§ 371 Date: Aug. 28, 2006

(30) Foreign Application Priority Data
Feb. 27, 2004 (CN) 200410025892.7

(51) Int. Cl. G01N 33/00

(52) U.S. Cl. 436/143

(57) ABSTRACT

An online monitoring method and device for a fossil fuel converter apparatus. The method monitors fuel compositions in real time by measuring operating data of the converter apparatus and comprises the following steps: determining reactant compositions and number of variables thereof; determining fuel compositions and number of variables thereof; determining compositions of incomplete products and number of variables thereof; determining relationship between the fuel compositions and calorific value; establishing an equation set involving the fuel compositions, the reactant compositions and the resultants compositions, according to energy balance relationship and material balance relationship in the combustion process; providing given conditions for independent relationships concerning variables in the above equation set; measuring boiler operating data and assigning the variables in the above equation set; and finding the solution to the equation set and obtaining real-time monitoring data of the converter apparatus.

### FIELD OF TECHNOLOGY

The present invention relates to online monitoring methods and devices for fossil fuel converter apparatus and is mainly used for the technical field of various fossil fuel converter apparatuses, e.g., large-scale power stations, to carry out online monitoring, simulation and optimal control on fuel property, efficiency and losses of the energy conversion process by means of computer and online operation data.

### BACKGROUND ART

Various fossil fuel converter apparatus need online monitoring of fuel property including fuel compositions and calorific value, efficiency and losses of the conversion process so as to optimize the control. The compositions and calorific value of a fossil fuel are naturally formed and thus are inevitably unstable and changing, and the efficiency and losses of a converter apparatus are also unstable and changing. It is necessary to grasp timely and precisely the data of the above two aspects, and all the data are required to be correspondent to and complied with each other, namely, a so-called closed solution, which plays an important role in application.

In the prior art, fossil fuels such as coal and oil have a problem that fuel property fails to be monitored online, thus efficiency and losses of the converter apparatus are difficult to be accurately measured. As to gas fuel, whose fuel property can be monitored online by means of a gas chromatograph, however, the efficiency and losses of the converter apparatus can neither be measured accurately nor will a closed solution be obtained.

The optimal control technique, for example, for generating units and portfolio of generator units of large-scale pulverized coal boilers, including boiler optimal control, is a worldwide difficult problem, wherein it is an essential obstacle to perform online and real-time monitoring on efficiency of pulverized coal boiler and coal quality. Due to the failure to grasp the boiler efficiency, the online monitoring and optimal control of the coal consumption of the whole generating unit and portfolio of generating units as well as costs for power generation lose the essential basis. Since the boiler efficiency monitoring needs coal quality data, if there is no real-time online monitoring of coal quality, the boiler efficiency monitoring cannot be realized.

Presently, the technique for boiler efficiency online monitoring in the world still depends on coal quality lab test data. Since the coal quality lab test is impossible to be in real-time and correspondent, the error range of boiler efficiency monitoring has reached ±2% and goes beyond the practical changing scope of boiler efficiency, which scope is deemed usually to be only ±1.5% by the applicant.

Therefore, the key of boiler efficiency monitoring lies in coal quality monitoring. The technique for coal quality online monitoring on a coal conveying belt in the fuel transport workshop by means of nuclear technique has been put into application only in very few power stations. However, this technique has high application costs including nuclear security costs, purchase costs and maintenance costs and has a poor response time, so there is no application example on the coal feeders of generating units. Meanwhile, since coal in the hopper does not move in the way of first-in-first-out, thus the output sequence from the coal conveying belt to the coal feeder becomes undetermined; the problem of online monitoring boiler efficiency and coal quality cannot be solved.

Chinese Patent No. 99112866.4 proposed a solving method for online analyzing boiler efficiency by means of computer software on the basis of some boiler operation data and has intended to find a method of calculating boiler efficiency through a particular deduction process. However, for an essential error in the deduction process, the result of loss of unburned carbon, which was calculated by the patent method, will have serious departure from the actual result.

Chinese Patent, No. 02110116.7 proposed a method for obtaining combined solution for the online ultimate analysis and efficiency analysis during boiler combustion by means of computer software on the basis of some boiler operation data. According to the research of the applicant, in the method as provided by the above-stated technique, the number of independent variables of the mathematical model is greater than the number of independent given conditions, thus the solutions defined by the boiler operation data are not unique, and the obtained solutions of coal quality and efficiency fail to represent the practical values. Therefore, this method failed to practically solve the problem of online monitoring of coal quality in pulverized coal boiler and has no applicability. Thus, this technical solution is invalid.

As a mathematical object, pulverized coal boiler combustion involves multiple variables, layers and subjects and is interdisciplinary, in engineering and in practice. It is a long standing formidable task for any organizations and personnel or the whole society to conduct a completely clear research on the object. The prior art took the practically unbalanced boiler process as an approximately balanced object so as to analyze boiler efficiency or coal quality through energy balance and material balance. This direction is considered to be correct by the applicant. However, the prior art failed to build up an integrated and penetrating theory. The essential reason for the invalidation of the prior art lies in the failure to theoretically reach an overall correct understanding of balanced mathematical modeling of boiler combustion, realize the correct method for establishing equation set, judge the number of independent variables and find the conditions for obtaining accurately correct solutions. Accordingly, a valid mathematical model and workable solutions fail to be established, thereby failing to form a valid research result which can be understood and acknowledged by those skilled in the art. To achieve the valid technical solution for online monitoring of boiler efficiency or coal quality, it is necessary to make in-depth, complete and long-term creative technical labor: including, far beyond the range involved by those skilled in the art, comprehensively understanding theories of boiler combustion, heat transfer, coal quality and mathematics and all the related engineering practices, so as to form complete and penetrating theories; and also including the establishment of valid mathematical modeling and workable solutions based on these theories. This is just what the prior art has not realized.

### SUMMARY OF THE INVENTION

In order to solve the above technical problem in the prior art, the present invention provides a set of online monitoring methods and devices for fossil fuel converter apparatus, wherein a balance parse of the converter apparatus is performed by means of a computer and operation data of the conversion process so as to realize a correct method for online monitoring of fossil fuel property and efficiency and losses of the converter apparatus.

Taking pulverized coal boiler as an example, the present invention performs a boiler balance parse by means of a computer so as to realize an entire set of correct methods for online monitoring of the efficiency of pulverized coal boiler and coal quality, i.e., the method set, according to different combinations of practical industrial conditions and boiler operation data, for carrying out real-time and online calculation of coal quality data based only on the boiler operation data or given data but independent of coal quality test data, and at the same time calculating validly the losses and efficiency of the boiler, including a plurality of boiler balance equation sets which are integrated and valid to the largest extent. Hence, it provides sufficiently effective performance monitoring support for the optimal control of boilers, generating units and portfolio of generating units.

According to a first aspect of the present invention, an online monitoring method for a fossil fuel converter apparatus is provided, which monitors fossil fuel compositions in real time by measuring operating data of the converter apparatus, characterized in that said online monitoring method comprises the following steps: a) determining reactant compositions and number of variables thereof; b) determining fossil fuel compositions and number of variables thereof; c) determining compositions of incomplete resultants and number of variables thereof; d) determining relationship between the fossil fuel compositions and calorific value; e) establishing a set of equations concerning to fossil fuel compositions, reactant compositions and resultant compositions, based on an energy balance relationship and a material balance relationship of the combustion process; f) providing given conditions concerning to independent relationships of variables for the set of equations; g) measuring boiler operating data and assigning values to the variables in the above equation set, wherein the sum of the number of the assigned variables and the number of the above given conditions are equal to the sum of the number of variables of the reactant compositions, number of variables of fossil fuel compositions and number of variables of incomplete resultants compositions, so as to achieve a positive definite condition of the equation set; and h) finding the solution to the equation set and obtaining real-time monitoring data of the fossil fuel converter apparatus.

According to a second aspect of the present invention, an online monitoring method for a fossil fuel converter apparatus is provided, wherein said fossil fuel defined in the first aspect of the present invention is coal.

According to a third aspect of the present invention, an online monitoring method for a fossil fuel converter apparatus is provided, wherein said fossil fuel converter apparatus defined in the second aspect of the present invention is a coal-fired boiler and the reactant is air, and in step (a), the compositions of air include O₂, N₂ and moisture of a constant proportion, and the micro contents including H₂, CO₂ and Argon is set to be zero; in step (c), the change of ash ratio and change of carbon content of slag is set to be zero, and nitrogen oxide content of flue gas is set to be zero; in step (b), ultimate analysis compositions of coal is determined to be C, H, O, N, S, M and A; in step d), said relationship is a Mendeleev's equation, or an empirical formula of calorific value of coal, which is expressed by the above elementary compositions; in step e), according to the energy balance relationship in the combustion process: the total calorific value of coal should be equal to the sum of boiler heat absorption and losses, and the pulverizing system has a thermal balance; wherein the material balance relationship is as follows: element C content is equal to C content of CO₂, CO in flue gas and of the unburned carbon in ash and slag; element S content is equal to S content of SO₂ in flue gas; the mass of moisture in flue gas is equal to the sum of H₂O produced from H combustion, H₂O in air and M of coal; the sum of O₂ content of air and the amount of O₂ produced from element 0 of coal should be equal to the sum of the amount of O₂ consumed by the oxidation of SO₂, CO₂ and CO, the amount of O₂ used for H combustion and the amount of the remaining O₂ in fuel gas; the sum of N₂ content of air and the amount of N₂ generated by element N of coal should be equal to N₂ content in fuel gas; the sum of boiler ash and slag is equal to A content of coal; and establishing an equation set; in step f), said given conditions are: regressive equation, empirical formula or set relationship between compositions of elements H and N; regressive equation, empirical formula or set relationship between compositions of elements C and O; and regressive equation, empirical formula or set relationship between compositions of elements C and H; in step g), the boiler operating data are measured to form the restricting conditions for the equation set so as to realize positive definite conditions of the equation set and the positive definite conditions of the equation set are selected from the following restricting conditions for the equation set formed by measuring boiler operating data: the amount of boiler heat absorption calculated according to the measured boiler steam/water parameters; total moisture M of coal calculated according to the coal flow-rate, air flow-rate and temperatures of each pulverizing system; total amount of coal feeding; total air input; amount of dry flue gas; amount of fly-ash and slag or ash content of coal; Carbon content of fly-ash; SO₂ content of dry flue gas; O₂ content of dry flue gas; CO content of dry flue gas; CO₂ content of dry flue gas; N₂ content of dry flue gas; and H₂O content of flue gas.

According to a fourth aspect of the present invention, an online monitoring method for a fossil fuel converter apparatus is provided, wherein in the third aspect of the present invention: in step g), said boiler operating data is measured to form the restricting conditions for the equation set so as to realize positive definite conditions of the equation set and the positive definite conditions are selected from the following restricting conditions for the equation set formed by measuring boiler operating data: the amount of boiler heat absorption calculated according to the measured boiler steam/water parameters; total moisture M of coal calculated according to the coal flow-rate, air flow-rate and temperature of the pulverizing system; total amount of coal feeding; total air input; amount of dry flue gas; amount of fly-ash and slag or ash content of coal; Carbon content of fly-ash; SO₂ content of dry flue gas; O₂ content of dry flue gas; CO content of dry flue gas; CO₂ content of dry flue gas; N₂ content of dry flue gas; and H₂O content of flue gas; according to the number of given conditions, measurement restricting conditions which are independent of the given conditions and independent of each other are selected from the measurement restricting conditions so that the total number of given conditions and measurement restricting conditions reaches ten.

According to a fifth aspect of the present invention, an online monitoring method for a fossil fuel converter apparatus is provided, wherein in the fourth aspect of the present invention: regarding a direct-firing pulverized coal boiler, said given conditions select two from the following three items: regressive equation, empirical formula or set relationship between compositions of elements H and N; regressive equation, empirical formula or set relationship between compositions of elements C and O; and regressive equation, empirical formula or set relationship between compositions of elements C and H; and the restricting conditions formed by the measured boiler operating data select the following seven items: the amount of boiler heat absorption calculated according to the measured boiler steam/water parameters; total moisture M of coal calculated according to the coal flow-rate, air flow-rate and temperature of the pulverizing system; total amount of coal feeding; total air input; SO₂ content of dry flue gas; O₂ content of dry flue gas; CO content of dry flue gas; and one of the following items is selected: CO₂ content of dry flue gas; N₂ content of dry flue gas; and H₂O content of flue gas; amount of dry flue gas; and Carbon content of fly-ash.

According to a sixth aspect of the present invention, an online monitoring method for a fossil fuel converter apparatus is provided, wherein in the fourth aspect of the present invention: regarding a middle-storage pulverized coal boiler, said given conditions select two from the following three items: regressive equation, empirical formula or set relationship between compositions of elements H and N; regressive equation, empirical formula or set relationship between compositions of elements C and O; and

regressive equation, empirical formula or set relationship between compositions of elements C and H; and the restricting conditions formed by the measured boiler operating data select the following seven items: the amount of boiler heat absorption calculated according to the measured boiler steam/water parameters; total moisture M of coal calculated according to the coal flow-rate, air flow-rate and temperature of the pulverizing system; SO₂ content of dry flue gas; O₂ content of dry flue gas; CO content of dry flue gas;

and three of the following items are selected: CO₂ content of dry flue gas; N₂ content of dry flue gas; and H₂O content of flue gas; amount of dry flue gas;

and Carbon content of fly-ash.

According to a seventh aspect of the present invention, an online monitoring method for a fossil fuel converter apparatus is provided, wherein in the third aspect of the present invention, regarding bituminous coal, the regress equation, empirical formula or set relationship of element compositions selects: regressive equation between compositions of elements C and O; and regressive equation between compositions of elements H and N.

According to an eighth aspect of the present invention, an online monitoring method for a fossil fuel converter apparatus is provided, wherein in the third aspect of the present invention, regarding anthracite, the regress equation, empirical formula or set relationship of element compositions selects: regressive equation between compositions of elements C and N; and regressive equation between compositions of elements H and N.

According to a ninth aspect of the present invention, an online monitoring method for a fossil fuel converter apparatus is provided, wherein in the third aspect of the present invention, regarding brown coal, the regress equation, empirical formula or set relationship of element compositions selects: regressive equation between compositions of elements C and O; and

regressive equation between compositions of elements H and N.

According to a tenth aspect of the present invention, an online monitoring method for a fossil fuel converter apparatus is provided, wherein in the third aspect of the present invention, a proximate analysis result of volatile matter of coal is found according to the regressive equation or an empirical formula of elementary constitution of coal and volatile matter.

According to an eleventh aspect of the present invention, an online monitoring method for a fossil fuel converter apparatus is provided, wherein in the third aspect of the present invention, the following given conditions can be added to said equation set: a proportion between the content of any two of elements C, H, O, N and S in the ash and moisture free base of coal is relatively stable; amount of air necessary for combustion of each kilogram of ash and moisture free base; amount of dry flue gas generated from the combustion of each kilogram of ash and moisture free base; amount of heat generated from the combustion of each kilogram of ash and moisture free base; hydrogen content necessary for the combustion of each kilogram of ash and moisture free base.

According to a twelfth aspect of the present invention, an online monitoring method for a fossil fuel converter apparatus is provided, wherein in the third or eleventh aspect of the present invention: said forming the restricting conditions for the equation set by measuring boiler operating data so as to realize positive definite conditions of the equation set including:

selecting six of the following restricting conditions for the equation set formed by measuring boiler operating data: the amount of boiler heat absorption calculated according to the measured boiler steam/water parameters; total moisture M of coal calculated according to the coal flow-rate, air flow-rate and temperature of the pulverizing system; total amount of coal feeding; total air input; amount of dry flue gas; amount of fly-ash and slag or ash content of coal; Carbon content of fly-ash; O₂ content of dry flue gas; CO content of dry flue gas; and H₂O content of flue gas.

According to a thirteenth aspect of the present invention, an online monitoring method for a fossil fuel converter apparatus is provided, wherein in the twelfth aspect of the present invention: regarding the direct-firing pulverized coal boiler, said restricting conditions formed by measuring boiler operating data select the following six items: the amount of boiler heat absorption calculated according to the measured boiler steam/water parameters; total moisture M of coal calculated according to the coal flow-rate, air flow-rate and temperature of the pulverizing system; total amount of coal feeding; total air input; O₂ content of dry flue gas; CO content of dry flue gas.

According to a fourteenth aspect of the present invention, an online monitoring method for a fossil fuel converter apparatus is provided, wherein in the twelfth aspect of the present invention, regarding the middle-storage pulverized coal boiler, said restricting conditions formed by measuring boiler operating data select the following six items: the amount of boiler heat absorption calculated according to the measured boiler steam/water parameters; total moisture M of coal calculated according to the coal flow-rate, air flow-rate and temperature of the pulverizing system; O₂ content of dry flue gas; CO content of dry flue gas; and H₂O content of flue gas; and amount of dry flue gas.

According to a fifteenth aspect of the present invention, an online monitoring method for a fossil fuel converter apparatus is provided, wherein in the first aspect of the present invention, said fossil fuel is fuel gas.

According to a sixteenth aspect of the present invention, an online monitoring method for a fossil fuel converter apparatus is provided, wherein in the first aspect of the present invention, said fossil fuel is fuel oil.

According to a seventeenth aspect of the present invention, an online monitoring device for a fossil fuel converter apparatus is provided, the apparatus comprises a computer, a data input passage for collecting converter apparatus operating data, and an output data display/output passage, for monitoring a fossil fuel converter apparatus in real-time, characterized in that, said online monitoring apparatus comprises: means for storing reactant compositions, fossil fuel compositions, resultant compositions, and relationship between the fossil fuel compositions and calorific value; means for storing an equation set involving the fossil fuel compositions, the reactant compositions and the resultant compositions, according to energy balance relationship and material balance relationship in the combustion process;

means for providing given conditions for use of the above equation set, wherein said given conditions are equations concerning variable relationship in the above equation set; means for, according to measured boiler operating data, assigning the variables in the above equation set to form positive definite conditions of the equation set, wherein the sum of the number of the assigned variables and the number of the above given conditions are equal to the sum of the number of variables of the reactant compositions, number of variables of fossil fuel compositions and number of variables of incomplete resultants compositions; and means for finding the solution to the equation set to obtain real-time monitoring data of the fossil fuel converter apparatus.

According to an eighteenth aspect of the present invention, an online monitoring device for a fossil fuel converter apparatus is provided, wherein said fossil fuel converter apparatus in the seventeenth aspect of the present invention can be selected from the group consisting of: pulverized coal boiler, circulating fluidized bed boiler, integrated gasification combined circulating power generator, gas-turbine combined circulating power generator, and fuel cells.

The present invention has the following advantages and produces the following positive effects:

The present invention realizes a direct online monitoring of fuel compositions, calorific value and efficiencies and losses of a converter apparatus by using practical operation parameters of the converter apparatus, and all the data obtained are accurate and closed. Meanwhile, the combinations of applied practical operation parameters are greatly flexible, that is, the input or output material compositions can be selected at the user's discretion according to the practical conditions of the converter apparatus. The following is to prove the positive definite conditions:

In a fossil fuel converter apparatus, the number of variables of a simultaneous equation set should be the number of independent equations plus the number of independent variables. In order to realize the positive definite solution of the equation set, it is necessary to assign the variables of the equation set or to give other independent relationships, and the total number of the assigned values and independent relationships should be the number of the independent variables. The number of independent variables should be the sum of the number of input fuel compositions, the number of variables of reactant (e.g., air, desulphurizer, H₂, etc.) and the number of incomplete combustible compositions in the output material.

The basic method for realizing the ultimate analysis model in the third aspect of the present invention is analyzed as a reference example.

The input to a boiler includes coal and air, and since the change of air component proportion is ignored, that is, air is an independent variable and the coal compositions contain C, H, O, N, S, M and A, thus, the boiler material input compositions have a total number of 8 independent variables; the material compositions output from the boiler are analyzed as follows:

Element C partially produces the unburned carbon in ash and slag;

Element C produces CO due to partially incomplete combustion;

Element C content is equal to the C content in CO₂ and CO in the flue gas and unburned carbon in the ash and slag;

Element S content is equal to the S content in SO₂ in the flue gas;

The mass of moisture in the flue gas is equal to the sum of H₂O produced from H combustion, H₂O of air and M of coal;

The sum of O₂ content of air and the amount of O₂ produced from element O in the coal should be equal to the sum of the amount of O₂ consumed by the oxidation of SO₂, CO₂ and CO, the amount of O₂ produced from H combustion, and the amount of O₂ remained in flue gas;

The sum of N₂ content of air and the amount of N₂ produced from element N of coal should be equal to the N₂ content of flue gas; and

The sum of the boiler ash and slag is equal to A content of coal.

It can be seen that, in the combustion of the boiler input material compositions, only the process in which element C partially produces the unburned carbon in ash and the process in which element C is partially combusted to produce CO are undetermined, so the boiler output material compositions only have two independent variables.

Since boiler heat input and every loss can be expressed by material compositions (and temperature, which must be measured), the number of independent variables of the boiler input and output material is the number of independent variables of the boiler. With regard to this example, the boiler has 10 independent variables. The total number of variables of a boiler simultaneous equation set is the number of independent equations plus the number of independent variables. In order to be positive definite, the equation set should have ten restricting conditions, that is, assigning the variables or giving independent relationships other than the above independent equations. The fourth aspect of the present invention gives 10 independent restricting conditions, which is the sum of given conditions and measured conditions. Hence, the positive definite conditions are proved.

In a complicated or simplified situation, the number of independent known conditions for realizing a positive definite condition can be deduced according to the increased or decreased number of independent variables. Take a basic method for realizing the proximate analysis model as defined in the eleventh aspect of the present invention as an example, the compositions of elements C, H, O, N and S in the coal model have a relatively constant proportion, and the number of independent variables is decreased from 10 to 6. Therefore, it is only necessary to have 6 independent known conditions of the positive definite condition. Thus, the equation set as defined in the twelfth aspect of the present invention is also positive definite.

The present invention essentially discloses the complete constitution and the number of independent variables of a combustion balance mathematical model for a pulverized coal boiler, so that a correct method for accurately monitoring boiler efficiency and coal quality is realized in any simplified condition and any coal composition model.

The method for realizing the present invention presents the basic methods including an ultimate analysis model and a proximate analysis model, respectively, and these methods have distinct applicability. The basic methods based on the present invention comprise a set of correct methods for online monitoring pulverized coal boiler efficiency and coal quality, namely, a set of methods for realizing a real-time and online calculation of coal quality data and effectively calculating the boiler losses and efficiencies according to different boiler operation data obtained from different industrial practical conditions and to the precise mathematical model which is multiform, systemized, complete and matching practical condition to the largest extent, without depending on coal lab test data but only based on the boiler operation data or given data, so as to provide sufficiently effective measurement supports for optimal control of boilers, machine sets and portfolio of generating units.

The present invention not only realizes an engineering workable solution, in which an integrated and precise mathematical model is used to obtain the unique solution of as-fired coal quality and boiler efficiency, but also has completely and thoroughly responded and solved the problem of real-time online monitoring of coal quality and boiler efficiency, theoretically and practically. Furthermore, the solutions set forth in the present invention can be used to perform a real-time online monitoring of coal quality and boiler efficiency under all kinds of different known conditions of boiler operation data. The different known conditions have thousands of complete combinations, so the engineering modes for realizing the present invention are countless.

With regard to the practical engineering application and theoretical study, the present invention develops an important and universal industrial standard method. Since the present invention sets forth a plurality of practical solutions based on the same theory, the specific solutions are greatly flexible in the practical applications with respect to the different conditions of targeted objects.

The different methods according to the present invention can be simultaneously put into use, and the results can be compared and analyzed, checked corrected, or averaged.

The present invention can establish a complete and precise boiler simultaneous equation set, having taken into consideration the influences of CO, mechanical unburned carbon, etc. on component balance and energy balance and formed an accurate mathematical model for boiler combustion, so as to obtain the better ultimate analysis result of coal. Since the boiler efficiency is calculated on the basis of the ultimate analysis result of coal, the calculation of the boiler losses and efficiencies in the present invention is closer to reality. Since the result of element C content is closer to reality, the volatile matter obtained according to the empirical formula has practical application value. The volatile matter, stack loss, loss due to incomplete combustion obtained according to the present invention can be used to directly guide the boiler combustion adjustment. The boiler efficiency calculation result obtained according to the present invention can be used to evaluate the boiler operation quality and diagnose the converter apparatus status.

Since the data obtained in the present invention is real-time, representative and real, the present invention not only realizes a real-time online monitoring of boiler efficiency and coal quality, but also accurately realizes a real-time online monitoring of coal consumption and costs for power generation of generating units.

The present invention covers the simplified and approximate solutions in any presumed conditions. For example, to ignore CO content in the flue gas or the unburned carbon in the ash or slag, it is only necessary to presume that the measured data is zero or a constant. For simplified stack loss calculation, just presume that the specific heat values of various gases are constants. To not take into consideration the moisture of air, just presume the moisture of air to be zero.

The present invention introduces coal composition modeling as independent given conditions, and these models take advantage of the research achievement of the coal industry thus have a sound scientific basis. In the previous research achievement, coal is considered to be plant fossil, wherein the ash content A thereof is associated with the coal mine and the exploitation position, and the moisture M is associated with storage and transportation, so the property of coal in the same coal mine is markedly changeable; sulphur S content is associated with the minerals during the submerge of sea water, so sulphur content of coal is greatly different depending on different mines and is random; C, H, O and N compositions are formed by the plant corrosion and their relative contents are restricted by coal age. The age of brown coal, bituminous coal, lean coal and anthracite varies from short to long, and the regressive equations of different types of coal are obtained according to the large number of coal quality test results and the regressive equation can reflect the regularity of one type of coal. Therefore, the ultimate analysis model method provided in the present invention can effectively online monitor the quality of one type of coal from many coal mines, and the proximate analysis model method satisfies the change of moisture and ash content of coal provided by one coal mine.

The coal quality monitoring performed by the present invention is in a summed manner, so the coal quality divergence reflected in the statistic analysis of the coal quality lab test will be distinctly reduced, the correlation of the regressive equation is better than that in the testing result, and correlation coefficients will be markedly improved over those given in the embodiments of the present invention, and therefore, this coal quality simulation technique has good realness. In addition, the coal quality data in terms of boiler combustion should be overall averaged data per se, so the result of the present invention is more representative than the lab test result. Since the boiler operation is continuously online monitored, the results of present invention have the best real-time nature in the actual sense.

One important advantage of the present invention is that the result can be improved with system calibration. The system calibration refers to obtaining coal quality data through lab tests when the as-fired coal quality is stable, wherein the quality data can be used to calibrate the empirical equation relating to the coal quality and can also be used as a given value to be input to a mathematical model to back-calculate the boiler operation data, and then the found boiler operation data is calibrated with the actual operation data. After the system calibration, the measurement result of the present invention has a higher engineering accuracy. Regarding the case where the ultimate analysis model solution method adopts the Mendeleev's equation as an equation for calculating the calorific value of coal, since this equation itself is a test standard for the calorific value of coal or elementary component test, the errors of boiler efficiency and coal quality realized by the present invention can be substantially ignored in engineering practice.

The present invention successfully introduces the numerical value simulation technique into the boiler efficiency and coal quality online monitoring and analyzing field. The boiler efficiency and coal quality online monitoring system realized according to the present invention is usually called a Boiler Efficiency and Coal Online Simulator, which is shortly referred to as BECos.

The errors in the prior art include method errors, measurement errors and modeling errors. The method errors include the errors resulting from sampling and testing or indefinite equation sets. The measurement errors are monitoring result errors resulting from measurement data errors. The modeling errors include monitoring result errors resulting from simplified conditions, given conditions and approximate factors of equations.

The present invention only has measurement errors and modeling errors. Compared with the prior art, the present invention eliminates serious method errors resulting from sampling and testing or indefinite equation sets or the like. In the modeling errors, the errors resulting from approximate factors of equations include that the heat radiation loss equation adopts the ratio of boiler rated load to actual load to reflect loss due to heat radiation and the like, and the errors resulting from given conditions include errors between given conditions of coal compositions and actual coal compositions. The measurement errors can be reduced through the measurement calibration and system calibration at each measurement point. The errors can be further reduced and the accuracy can be further improved in the present invention by minimizing the equation approximation, reducing simplified equations and given conditions and correspondingly increasing measurement restricting conditions, and reducing measurement errors.

By precisely implementing the present invention and normally maintaining the accuracy of measurement points of the present invention, the accurate real-time and on-line monitoring of boiler efficiency and coal quality can be assured. The present invention can provide reliable monitoring data for boiler diagnosis, control basis, cost tracking, coal quality checking and management reference, and can also replace the traditional boiler efficiency tests and routine coal quality lab tests which have consumed great manpower and material resources.

From theory to practice, the present invention comprehensively answers and solves the problem of real-time and on-line monitoring of a fossil fuel converter apparatus and realizes an engineering solution for obtaining one unique solution of the fossil fuel compositions, calorific value, converter apparatus efficiency and losses by using a complete and precise mathematic model, and the presented solution can be used for monitoring under various given conditions. Since the data obtained in the present invention are real-time, representative and real, the present invention realizes a significantly substantive breakthrough in the technique of monitoring and optimal control system of large-scale fossil fuel power stations.

### BEST MODES FOR CARRYING OUT THE PRESENT INVENTION

The present invention provides a set of methods for jointly solving and integrated online monitoring fossil fuel material and converter apparatus losses in real time. The modes for realizing the present invention comprise those for use of offline simulation, online simulation and online monitoring. Online monitoring is the most important mode for realizing the present invention.

The preferred mode for realizing the device of the present invention using the online monitoring mode is as follows: using an independent computer as a station to be connected to a DCS LAN network, obtaining all the measurement data necessary for measuring restricting conditions by implementing a database, performing a computation once every ten seconds, including average filtering input data in the last ten seconds, solving equation sets and sending computation results, i.e., monitoring data, to the real-time database.

Using the preferred mode of the method of the present invention should take into account the following three factors so as to select the most applicable technical solution in the method set provided by the present invention: e.g., pulverized coal boiler.

1) Conditions of Devices under Test and Original Measuring Systems

Direct-firing: usually, the total amount of fed coal and the total air input (including measuring air leakage) can be solved by using the original measurement point;

Middle-storage: usually, the effective measurement data regarding the total amount of coal input and the total air input cannot be obtained, and it is necessary to use more measurements on the post combustion variables so as to guarantee the same number of measurement restricting conditions.

Generating unit capacity: the large generating unit can tend to use the more measurement restricting conditions, while the small generator group can tend to choose more given conditions.

It's preferable to use the existing measurement point of the original measurement system to reach the number of restricting conditions. For example, an original flue gas online monitoring device probably has online monitoring of CO, CO₂ and SO₂ in the flue gas, and velocity of flue gas. However, some measurement points are located behind the desulphurization process, and if it is possible to ignore the influence of the desulphurization process or back-calculate the flue gas data and meet the requirements for accuracy, then the original measurement system can be adopted.

2) Accuracy requirement for the following objects of the present invention:

The accuracy necessary requirements for the following objectives vary from high to low:

Boiler diagnosis;

Control basis;

Costs tracking;

Coal quality checking; and

Management reference;

3) When necessary, it should be considered to add boiler measurement items, i.e., measurement restricting conditions, to realize the monitoring object:

The following measurement items relate to more specialized measurement practices, which the original measuring system may not have:

Online monitoring Carbon content of fly-ash;

Online monitoring of H₂O in flue gas;

Online monitoring of CO in flue gas;

Online monitoring of CO₂ in flue gas;

Online monitoring of SO₂ in flue gas;

Flue gas monitoring differs from environment monitoring in that the location of measurement point is before the flue gas processing device.

In case of high accuracy is required, the ultimate analysis coal quality model solutions should be adopted, namely, said solutions in the third to tenth aspects of the method of the present invention; while in case of low accuracy is acceptable, the proximate analysis coal quality model solutions should be adopted, namely, said solutions in the eleventh to fourteenth aspects of the method of the present invention.

All the modes for realizing the present invention are the problems of establishing a boiler combustion balance equation set and selecting known variables and unknown variables. Through the description and five embodiments as provided, those skilled in the art are capable of carrying out selection and implementation according to industrial on-site conditions and requirements.

On the component regressive equation of coal quality ultimate analysis:

The regressive equation of compositions between elements C and O is Oₐᵣ=A₁Cₐᵣ+B₁, and it is applicable to bituminous coal and brown coal, wherein regarding bituminous coal, A₁=0.9, B₁=85.0 and the relevant coefficient γ=0.98; regarding brown coal, A₁=0.84, B₁=80.38 and the relevant coefficient γ=0.95.

The regressive equation between compositions of said elements H and N is Nₐᵣ=A₂Hₐᵣ+B₂, wherein A₂=0.3, B₂=0.

The regressive equation between compositions of elements C and H is Hₐᵣ=A₃Cₐᵣ+B₃, and it is applicable to anthracite and bituminous coal, wherein regarding anthracite, A₃=0.448, B₃=44.73, and the relevant coefficient γ=0.83; regarding bituminous coal, A₃=0.24, B₃=26.1, and the relevant coefficient γ=0.72.

The regressive equation between compositions of volatile matter V and element C is Vₐᵣ=A₄Cₐᵣ+B₄, wherein regarding bituminous coal, A₄=2.52, B₄=247.8, and the relevant coefficient γ=0.86; regarding anthracite, A₄=1.087, B₄=107.5, and the relevant coefficient γ=0.86; regarding brown coal, A₄=1.14, B₄=129.9, and the relevant coefficient γ=0.74.

In the embodiment of the present invention, the input temperature of raw material and air is t₀, flue gas temperature thereof is θ_{py}, and the slag temperature and specific heat are constants.

### Embodiment 1

According to the ultimate model solution of the present invention, the embodiment adopts the following ten conditions:

1) The total element calorific value should be equal to the sum of the amount of boiler heat absorption and all the losses. See equation (12).

2) The pulverizing system has a heat balance. Calculate Mₐᵣ.

3) The total amount of input coal is equal to the sum amount of elements, H₂O and ash. See equation (2).

4) Practically measure SO₂ in flue gas and obtain a corresponding equation concerning S of coal and SO₂ in flue gas. See equation (5).

5) Regressive equation or empirical equation between compositions of elements (e.g., H, N). See equation (4).

6) Regressive equation or empirical equation between compositions of elements (e.g., C, O). See equation (3).

7) Practically measure O₂ in flue gas and obtain corresponding equations represented by coal compositions. See equations (6), (7), (8) and (9).

8) Practically measure CO₂ in flue gas and obtain corresponding equations represented by coal compositions. See equations (10) and (11).

9) Practically measure CO in flue gas and obtain corresponding equations represented by coal compositions. See equations (7) and (10).

10) Practically measure C content in fly-ash and obtain a corresponding equation represented by coal compositions. See equation (8).

Total coal flow-rate B measured by the coal feeding system.

Main steam flow D, main steam temperature t, reheating steam flow D′, reheating inlet steam temperature t′, reheating outlet steam temperature t″, and other conventional heat power data are measured, and boiler heat absorption amount BQ₁ is calculated.

O₂, SO₂, CO₂ and CO obtained through flue gas measurement.

C_{fh} is obtained by measuring Carbon content of fly-ash,

According to the statistic or average value of fly-ash quotient α_{fh} and Carbon content C_{lz} of slag, calculating out unburned carbon,
C_{ub}=Aₐᵣ[(1−α_{fh}) C_{lz}/(1−C_{lz})+α_{fh}C_{fh}/(1−C_{fh})] (1)

According to coal elementary composition equation,
Cₐᵣ+Hₐᵣ+Oₐᵣ+Nₐᵣ+Sₐᵣ+Aₐᵣ=100−Mₐᵣ (2)

Empirical equation of element content of coal, regarding bituminous coal,
Oₐᵣ=A₁Cₐᵣ+B₁ (3)
A₁=0.9, B₁=85.0.
Nₐᵣ=A₂Hₐᵣ+B₂
A₂=0.3, B₂=0. (4)

Equation for calculating dry flue gas using S content
V_{gy}=100×0.007 Sₐᵣ/SO₂ (5)

Relationship between dry flue gas and air excess coefficient
V_{gy}=(V_{gy}⁰+(α−1) V_{gk}⁰) (6)

Wherein α is air excess coefficient (ratio of the total air amount to the amount of dry air calculated according to the actual burn-out carbon),
α=21/(21−79(O₂−0.5CO)/(100−(CO₂+SO₂+CO+O₂))) (7)

V_{gk}⁰ is the amount of dry air necessary for theoretical combustion as calculated based on the application of base element compositions, taking into account the mechanical unburned carbon,
V_{gk}⁰=0.0889(Cₐᵣ+0.375Sₐᵣ)+0.265Hₐᵣ−0.0333Oₐᵣ−0.0889C_{ub} (8)

V_{gy}⁰ is the amount of dry flue gas produced from theoretical combustion as calculated based on the application of base element compositions, taking into account the mechanical unburned carbon,
V_{gy}⁰=0.0889Cₐᵣ+0.03331Sₐᵣ+0.2094Hₐᵣ+0.008Nₐᵣ−0.0263Oₐᵣ−0.0889C_{ub} (9)

Relationship between flue gas compositions and fuel property coefficients:
CO₂+SO₂+O₂=21−β(CO₂+SO₂)−(0.605+β)CO (10)

Fuel property coefficient:
β=2.35(Hₐᵣ−0.126Oₐᵣ+0.038Nₐᵣ)/(Cₐᵣ−C_{ub}+0.375Sₐᵣ) (11)

Empirical equation between low-level calorific value and element content of coal:
Q_{ar,net}=339Cₐᵣ+1028Hₐᵣ−109(Oₐᵣ−Sₐᵣ)−25Mₐᵣ (12)

Moisture in flue gas,
V_{H2O}=0.0124Mₐᵣ+0.1118Hₐᵣ+0.0161αV_{gk}⁰ (13)

Volume of various flue gases:
V_{R02}=V_{gy}(CO₂+SO₂) (14)
V_{cO}=V_{gy}CO (15)
V₀₂=V_{gy}O₂ (16)
V_{N2}=V_{gy}(100−(CO₂+SO₂+CO+O₂)) (17)

Equations of boiler efficiency and losses:

Boiler efficiency:
η=Q₁/Q_{ar,net} (18)

Stack loss:
q₂=[(V_{R02}C_{CO2}+V_{CO}C_{CO}+V_{N2}C_{N2}+V₀₂C₀₂+V_{H2O}C_{H2O}) θ_{py}−αV_{gk}⁰(C_{gk}+0.0161C_{H2O})t₀]/Q_{ar,net} (19)

Loss due to chemical incomplete combustion:
q₃=126.4V_{gy}CO/Q_{ar,net} (20)

Loss due to mechanical incomplete combustion
q₄=328.66C_{ub}/Q_{ar,net} (21)

Loss due to heat radiation:
q₅=qₑDₑ/D (22)

Loss due to physical heat of ash and slag:
q₆=Aₐᵣ[C_{fh}θ_{py}α_{fh}+554(1−α_{fh})]/100Q_{ar,net} (23)

Boiler heat balance equation:
η+q₂+q₃+q₄+q₅+q₆=1 (24)

The above 24 equations having 24 variables (Cₐᵣ, Hₐᵣ, Oₐᵣ, Nₐᵣ, Sₐᵣ, V_{gk}⁰, V_{gy}⁰, V_{gy}, α, β, C_{ub}, Aₐᵣ, V_{R02}, V_{N2}, V₀₂, V_{CO}, V_{H2O}, Q_{ar,net}, η, q₂, q₃, q₄, q₅, q₆) establish a boiler combustion simultaneous equation set. The method for solving the boiler combustion simultaneous equation set is as follows:

Take Aₐᵣ of a common coal type in practice as an initial value of the variable Aₐᵣ, and find the initial value (C_{ub})⁰ of C_{ub} according to equation (1)

Set δ=0.1, ξ=−1, μ=−0.5.

The following is a multi-degree gradual approximate iteration solving process, set Σq=η+q₂+q₃+q₄+q₅+q₆ and set the iteration degree as i:

Step 1: perform substitution of data of (Aₐᵣ)ⁱ in equation (1) to find the solution of (C_{ub})ⁱ.

Step 2: perform substitution of data of (C_{ub})ⁱ in equations (8) and (9).

Step 3: perform substitution of equations (5), (7), (8) and (9) in equation (6) and find the solution of β according to equation (10), and perform substitution of β in equation (11), and this equation (11) and equations (2), (3), (4) and (6) form a simple linear equation in five unknowns set and directly find the solutions of (Cₐᵣ)ⁱ, (Hₐᵣ)ⁱ, (Oₐᵣ)ⁱ, (Nₐᵣ)ⁱ, and (Sₐᵣ)ⁱ.

Step 4: perform substitution of data of (Cₐᵣ)ⁱ, (Hₐᵣ)ⁱ, (Oₐᵣ)ⁱ, (Nₐᵣ)ⁱ, (Sₐᵣ)ⁱ in equations (6), (9), (12) and (23) to find the solutions of (Q_{ar,net})ⁱ, (q₁)ⁱ, (q₂)ⁱ, (q₃)ⁱ, (q₄)ⁱ, (q₅)ⁱ and (q₆)ⁱ.

Step 5: find the solution of Σq−1, and if |Σ−1|ⁱ≦0.001%, the solving process ends and Step 9 begins.

Step 6: if (Σq−1)ⁱ (Σ−1)ⁱ⁺¹30 0, |Σ−1|ⁱ−|Σq−1|ⁱ⁺¹<0, set ξ=1.

Step 7: if (Σq−1)ⁱ−(Σq−1)ⁱ⁺¹<0, δ=μδ.

Step 8: set (Aₐᵣ)ⁱ⁺¹=(Aₐᵣ)ⁱ+ξδ, and return to Step 1.

Step 9: the gradual approximate iteration solving process ends.

Volatile matter calculation:
Vₐᵣ=A₄Cₐᵣ+B₄, wherein regarding bituminous coal, A₄=−2.52, B₄=247.8.

Fixed carbon calculation:
C_{ar,GD}=100−(Vₐᵣ+Aₐᵣ+Mₐᵣ).

### Embodiment 2

According to the ultimate model solution of the present invention, the embodiment adopts the following ten conditions:

1) The total element calorific value should be equal to the sum of the amount of boiler heat absorption and all the losses. See equation (12).

2) The pulverizing system has a heat balance. Calculate Mₐᵣ.

3) The total amount of input coal is equal to the sum amount of elements, H₂O and ash. See equation (2).

4) Practically measure SO₂ in flue gas and obtain a corresponding equation containing S of coal and SO₂ in flue gas. See equation (5).

5) regressive equation or empirical equation between compositions of elements (e.g., H, N). See equation (4).

6) regressive equation or empirical equation between compositions of elements (e.g., C, H). See the following equation:
Hₐᵣ=A₃Cₐᵣ+B₃, wherein regarding anthracite, A₃=0.448, B₃=44.73.

7) Practically measure O₂ in flue gas and obtain corresponding equations represented by coal compositions. See equations (6), (7), (8) and (9).

8) Practically measure CO₂ in flue gas and obtain corresponding equations represented by coal compositions. See equations (10) and (11).

9) Practically measure CO in flue gas and obtain corresponding equations represented by coal compositions. See equations (7) and (10).

10) Practically measure Carbon content of fly-ash and obtain a corresponding equation represented by coal compositions. See equation (8).

Volatile matter calculation:
Vₐᵣ=A₄Cₐᵣ+B₄, wherein regarding anthracite, A₄=−1.087, B₄=107.5.

Fixed carbon calculation:
C_{ar,GD}=100−(Vₐᵣ+Aₐᵣ+Mₐᵣ).

Refer to Embodiment 1 for the solving process.

### Embodiment 3

According to the ultimate model solution of the present invention, the embodiment adopts the following ten conditions:

1) The total element calorific value should be equal to the sum of the amount of boiler heat absorption and all the losses. See equation (12).

2) The pulverizing system has a heat balance. Calculate Mₐᵣ.

3) The total amount of input coal is equal to the sum amount of elements, H₂O and ash. See equation (2).

4) Practically measure SO₂ in flue gas and obtain a corresponding equation containing S of coal and SO₂ in flue gas. See equation (5).

5) Regressive equation or empirical equation between compositions of elements (e.g., H, N). See equation (4).

6) Regressive equation or empirical equation between compositions of elements (e.g., C, O). See equation (3).

7) Practically measure O₂ in flue gas and obtain corresponding equations represented by coal compositions. See equations (6), (7), (8) and (9).

8) Practically measure the amount of fly-ash or ash of coal and obtain corresponding equations represented by coal compositions. See equation (2).

9) Practically measure CO in flue gas and obtain corresponding equations represented by coal compositions. See equations (7) and (10).

10) Practically measure C content of fly-ash and obtain a corresponding equation represented by coal compositions. See equation (8).

Refer to Embodiment 1 for the solving process.

Volatile matter calculation:
Vₐᵣ=A₄Cₐᵣ+B₄, wherein regarding bituminous coal, A₄=−2.52, B₄=247.8.

Fixed carbon calculation:
C_{ar,GD}=100−(Vₐᵣ+Aₐᵣ+Mₐᵣ).

Refer to Embodiment 1 for the solving process.

### Embodiment 4

According to the ultimate model solution of the present invention, the embodiment adopts the following ten conditions:

1) The total element calorific value should be equal to the sum of the amount of boiler heat absorption and all the losses. See equation (12).

2) The pulverizing system has a heat balance. Calculate Mₐᵣ.

3) The total amount of input coal is equal to the sum amount of elements, H₂O and ash. See equation (2).

4) Practically measure SO₂ in flue gas and obtain a corresponding equation containing S of coal and SO₂ in flue gas. See equation (5).

5) Regressive equation or empirical equation between compositions of elements (e.g., H, N). See equation (4).

6) Regressive equation or empirical equation between compositions of elements (e.g., C, O). See equation (3).

7) Practically measure O₂ in flue gas and obtain corresponding equations represented by coal compositions. See equations (6), (7), (8) and (9).

8) Regressive equation or empirical equation between compositions of elements (e.g., C, H). See the following equation:
Hₐᵣ=A₃Cₐᵣ+B₃, wherein regarding bituminous coal, A₃=−0.24, B₃=26.1.

9) Practically measure CO in flue gas and obtain corresponding equations represented by coal compositions. See equations (7) and (10).

10) Practically measure C content of fly-ash and obtain a corresponding equation represented by coal compositions. See equation (8).

Refer to Embodiment 1 for the solving process.

Volatile matter calculation:
Vₐᵣ=A₄Car+B₄, wherein regarding bituminous coal, A₄=−2.52, B₄=247.8.

Fixed carbon calculation:
C_{ar,GD}=100−(Vₐᵣ+Aₐᵣ+Mₐᵣ).

### Embodiment 5

According to the proximate model solution of the present invention, the embodiment adopts the following six conditions:

1) The total calorific value of coal should be equal to the sum of the amount of boiler heat absorption and all the losses. See equation (37).

2) The pulverizing system has a heat balance. Calculate Mₐᵣ.

3) The total amount of input coal is equal to the sum amount of ash and moisture free base R, H₂O and ash. See equation (32).

4) Practically measure O₂ in flue gas and obtain corresponding equations represented by coal compositions. See equations (33), (34), (35) and (36).

5) Practically measure CO in flue gas and obtain loss of chemical incomplete combustion. see equation (41).

6) Practically measure Carbon content of fly-ash and obtain a corresponding equation represented by coal compositions. See equations (35) and (36).

According to the statistic rules or averages of fly-ash quotient α_{fh} and Carbon content C_{lZ} of slag, calculate out unburned carbon,
C_{ub}=Aₐᵣ[(1−α_{fh})C_{lz}/(1−C_{lz})+α_{fh}C_{fh}/(1−C_{fh})] (31)

According to coal element component equation,
R+Aₐᵣ=100−Mₐᵣ (32)

Relationship between dry flue gas and air excess coefficient
V_{gy}=(V_{gy}⁰+(α−1)V_{gk}⁰) (33)

Wherein α is air excess coefficient (ratio of the total air amount to the amount of dry air calculated for the actual burn-out carbon),
α=21/(21−O₂) (34)

V_{gk}⁰ is the amount of dry air necessary for theoretical combustion as calculated based on the application of element compositions, taking into account the mechanical unburned carbon,
V_{gk}⁰=K₁R−0.0889C_{ub} (35),

K₁ is the amount of air necessary for the combustion of per kilogram of ash and moisture free base,

V_{gy}⁰ is the amount of dry fuel gas produced from theoretical combustion as calculated based on the application of base element compositions, taking into account the mechanical unburned carbon,
V_{gy}⁰=K₂R−0.0889C_{ub} (36)

K₂ is the amount of dry flue gas necessary for the combustion of per kilogram of ash and moisture free base,

Empirical equation between low-level calorific value and element content of coal:
Q_{ar,net}=K₃R−25Mₐᵣ (37)

K₃ is the amount of heat generated from the combustion of per kilogram of ash and moisture free base,

Moisture of flue gas 12:
V_{H2O}=0.0124Mₐᵣ+0.1118K₄R+0.0161αV_{gk}⁰ (38)

K4 is Hydrogen content of per kilogram of ash and moisture free base,

Equations of boiler efficiency and losseses:

Boiler efficiency:
η=Q₁/Q_{ar,net} (39)

Stack loss:
q₂=[V_{gy}C_{gy}θ_{py}−αV_{gk}⁰(C_{gk}+0.0161C_{H2O})t₀]/Q_{ar,net} (40)

Loss due to chemical incomplete combustion:
q₃=126.4V_{gy}CO/Q_{ar,net} (41)

Loss due to mechanical incomplete combustion
q₄=328.66C_{ub}/Q_{ar,net} (42)

Loss due to heat radiation:
q₅=qₑDₑ/D (43)

Loss due to physical heat radiation of ash and slag:
q₆=Aₐᵣ[C_{fh}θ_{py}α_{fh}+554(1−α_{fh})]/100Q_{ar,net} (44)

Boiler heat balance equation:
η+q₂+q₃+q₄+q₅+q₆=1 (45)

The above 15 equations having 15 variables (R, V_{gk}⁰, V_{gy}⁰, V_{gy}, α, C_{ub}, Aₐᵣ, V_{H2O}, Q_{ar,net}, η, q₂, q₃, q₄, q₅, q₆) establish a boiler combustion simultaneous equation set.

Refer to Embodiment 1 for the solving process.

Volatile matter calculation:

Vₐᵣ=K₅R, wherein K5 is volatile matter content of per kilogram of ash and moisture free base.

As to different fossil fuels, such as oil and gas, their calorific values can both be expressed by equation (12), and the coefficients thereof can be adjusted as needed. Regarding the resultant of combustion, H₂ and hydrocarbon can be ignored. Thus, the present invention can be realized by using the same model and method as the above embodiments. When necessary, regarding the influences on efficiency caused by H₂ and hydrocarbon, the data of efficiency and calorific value of fuel obtained from the application of the present invention can be corrected according to the practically measured data. Regarding gas fuels, content and volume percentage of hydrocarbon, N₂, sulfureted hydrogen, CO₂ and other molecule compositions can be calculated through the requisite empirical relationship of hydrocarbon and other compositions and based on the element compositions calculated by the present invention.

Regarding different converter apparatuses, such as pulverized coal boiler, circulating fluidized bed (CFB) boiler, integrated gasification combined circulating power generator, gas-turbine combined circulating power generator, and fuel cells, their energy balance equations are established according to actual engineering data of the process.

As to the boiler combustion principles and calculating technique, the relevant content of Chapters 2 and 3 of Boiler Principles and Calculation (3^{rd} Edition) (FENG Junkai et al., Scientific Publishing House, 2003), which is the teaching material of Tsinghua University, can be consulted.

Regarding the measuring techniques for boiler operation parameters, flue gas and compositions of fly-ash and computer data acquisition system techniques, the relevant industrial standards, engineering technique materials and product materials can be applied through consultation, including the sufficient supplementary reference information which can be searched via internet.

Regarding the knowledge of component empirical equations of coal quality, it can be obtained from the application of materials of national raw coal minerals and coal types or test analysis statistics. For the materials of raw coal of China's coal mine, Coal Science (Geologic Publishing House, BAI Lingren, 1989) and other materials can be consulted.

The above techniques are the prior art which should be mastered by those skilled in the art or realized in daily work. However, since the present invention involves multiple subjects and majors and relates to the wide engineering techniques of pulverized coal boiler operation, monitoring and analysis, the applicant believes that the present invention probably needs to be co-realized by plural persons skilled in the art.

1. An online monitoring method for a fossil fuel converter apparatus, which monitors fossil fuel compositions in real time by measuring operating data of the converter apparatus, characterized in that said online monitoring method comprises the following steps:
a) Determining reactant compositions and number of variables thereof;
b) Determining fossil fuel compositions and number of variables thereof;
c) Determining compositions of incomplete products among resultants and number of variables thereof;
d) Determining relationship between the fossil fuel compositions and calorific value;
e) Establishing an equation set involving the fossil fuel compositions, the reactant compositions and the resultants compositions, according to energy balance relationship and material balance relationship in the combustion process;
f) Providing given conditions for use of the above equation set, wherein said given conditions are independent relationships concerning the variables in the above equation set;
g) Measuring boiler operating data and assigning the variables in the above equation set, wherein the sum of the number of the assigned variables and the number of the above given conditions are equal to the sum of the number of variables of the reactant compositions, number of variables of fossil fuel compositions and number of variables of incomplete products compositions, so as to achieve a positive definite condition of the equation set; and
h) Finding the solution to the equation set and obtaining real-time monitoring data of the fossil fuel converter apparatus.

2. The online monitoring method for a fossil fuel converter apparatus according to claim 1, characterized in that said fossil fuel is coal.

3. The online monitoring method for a fossil fuel converter apparatus according to claim 2, characterized in that said fossil fuel converter apparatus is a coal-fired boiler and the reactant is air, wherein,
In step (a), the compositions of air include O₂, N₂ and steam of a constant proportion, and the content of micro-gas including H₂, CO₂ and Argon is set to be zero;
In step (c), the change of ash ratio and change of carbon content of slag is set to be zero, and nitrogen oxide content of flue gas is set to be zero;
In step (b), ultimate analysis compositions of coal is determined to be C, H, O, N, S, M and A;
In step (d), said relationship is a Mendeleev's equation, or an empirical formula of calorific value of coal, which is expressed by the above elementary compositions;
In step e), according to the energy balance relationship in the combustion process:
The total calorific value of coal should be equal to the sum of boiler heat absorption and losses, and the pulverizing system has a thermal balance;
Wherein the material balance relationship is as follows:
Element C content is equal to C content of CO₂, CO in flue gas and of the unburned carbon in ash; element S content is equal to S content of SO₂ in flue gas; the mass of steam in flue gas is equal to the sum of H₂O produced from H combustion, H₂O in air and M of coal; the sum of O₂ content of air and the amount of O₂ produced from element O of coal should be equal to the sum of the amount of O₂ consumed by the oxidation of SO₂, CO₂ and CO, the amount of O₂ used for H combustion and the amount of the remaining O₂ in fuel gas; the sum of N₂ content of air and the amount of N₂ generated by element N of coal should be equal to N₂ content in fuel gas; the sum of boiler ash and slag is equal to A content of coal;
And establishing an equation set;
In step f), said given conditions are:
Regressive equation, empirical formula or set relationship between compositions of elements H and N;
Regressive equation, empirical formula or set relationship between compositions of elements C and O; and
Regressive equation, empirical formula or set relationship between compositions of elements C and H;
In step g), the boiler operating data are measured to form the restricting conditions for the equation set so as to realize positive definite conditions of the equation set and the positive definite conditions of the equation set are selected from the following restricting conditions for the equation set formed by measuring boiler operating data:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
Amount of dry flue gas;
Amount of fly-ash and slag or ash content of coal;
Carbon content of fly-ash;
SO₂ content of dry flue gas;
O₂ content of dry flue gas;
CO content of dry flue gas;
CO₂ content of dry flue gas;
N₂ content of dry flue gas; and
H₂O content of flue gas.

4. The online monitoring method for a fossil fuel converter apparatus according to claim 3, characterized in that, in step g), said boiler operating data is measured to form the restricting conditions for the equation set so as to realize positive definite conditions of the equation set and the positive definite conditions are selected from the following restricting conditions for the equation set formed by measuring boiler operating data:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
Amount of dry flue gas;
Amount of fly-ash and slag or ash content of coal;
Carbon content of fly-ash;
SO₂ content of dry flue gas;
O₂ content of dry flue gas;
CO content of dry flue gas;
CO₂ content of dry flue gas;
N₂ content of dry flue gas; and
H₂O content of flue gas;
According to the number of given conditions, measurement restricting conditions which are independent of the given conditions and independent of each other are selected from the measurement restricting conditions so that the total number of given conditions and measurement restricting conditions reaches ten.

5. The online monitoring method for a fossil fuel converter apparatus according to claim 4, characterized in that, regarding a direct-firing pulverized coal boiler, said given conditions select two from the following three items:
Regressive equation, empirical formula or set relationship between compositions of elements H and N;
Regressive equation, empirical formula or set relationship between compositions of elements C and O; and
regressive equation, empirical formula or set relationship between compositions of elements C and H; and
The restricting conditions formed by the measured boiler operating data select the following seven items:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
SO₂ content of dry flue gas;
O₂ content of dry flue gas;
CO content of dry flue gas; and
one of the following items is selected:
CO₂ content of dry flue gas;
N₂ content of dry flue gas; and
H₂O content of flue gas;
Amount of dry flue gas; and
Carbon content of fly-ash.

6. The online monitoring method for a fossil fuel converter apparatus according to claim 4, characterized in that, regarding a middle-storage pulverized coal boiler, said given conditions select two from the following three items:
Regressive equation, empirical formula or set relationship between compositions of elements H and N;
Regressive equation, empirical formula or set relationship between compositions of elements C and O; and
Regressive equation, empirical formula or set relationship between compositions of elements C and H; and
The restricting conditions formed by the measured boiler operating data select the following seven items:
The amount of boiler heat absorption calculated according to the measured boiler steam/water parameters;
Total moisture M of coal calculated according to the coal flow-rate, air flow-rate and temperature of the pulverizing system;
SO₂ content of dry flue gas;
O₂ content of dry flue gas;
CO content of dry flue gas; and
Three of the following items are selected:
CO₂ content of dry flue gas;
N₂ content of dry flue gas; and
H₂O content of flue gas;
Amount of dry flue gas; and
Carbon content of fly-ash.

7. The online monitoring method for a fossil fuel converter apparatus according to claim 3, characterized in that, regarding bituminous coal, the regress equation, empirical formula or set relationship of element compositions selects:
Regressive equation between compositions of elements C and O; and
Regressive equation between compositions of elements H and N.

8. The online monitoring method for a fossil fuel converter apparatus according to claim 3, further characterized in that, regarding anthracite, the regress equation, empirical formula or set relationship of element compositions selects:
Regressive equation between compositions of elements C and N; and
Regressive equation between compositions of elements H and N.

9. An online monitoring method for a fossil fuel converter apparatus according to claim 3, further characterized in that, regarding brown coal, the regress equation, empirical formula or set relationship of element compositions selects:
Regressive equation between compositions of elements C and O; and
Regressive equation between compositions of elements H and N.

10. The online monitoring method for a fossil fuel converter apparatus according to claim 3, characterized in that, a proximate analysis result of volatile matter of coal is found according to then regressive equation or an empirical formula of elementary constitution of coal and volatile matter.

11. The online monitoring method for a fossil fuel converter apparatus according to claim 3, characterized in that,
The following given conditions can be added to said equation set:
A proportion between the content of any two of elements C, H, O, N and S in the ash and moisture free base of coal is relatively stable;
Amount of air necessary for combustion of each kilogram of ash and moisture free base;
Amount of dry flue gas generated from the combustion of each kilogram of ash and moisture free base;
Amount of heat generated from the combustion of each kilogram of ash and moisture free base; and
Hydrogen content necessary for the combustion of each kilogram of ash and moisture free base.

12. The online monitoring method for a fossil fuel converter apparatus according to claim 3, characterized in that,
Said forming the restricting conditions for the equation set by measuring boiler operating data so as to realize positive definite conditions of the equation set including:
Selecting six of the following restricting conditions for the equation set formed by measuring boiler operating data:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
Amount of dry flue gas;
Amount of fly-ash and slag or ash content of coal;
Carbon content of fly-ash;
O₂ content of dry flue gas;
CO content of dry flue gas; and
H₂O content of flue gas.

13. The online monitoring method for a fossil fuel converter apparatus according to claim 12, characterized in that, regarding the direct-firing pulverized coal boiler, said restricting conditions formed by measuring boiler operating data select the following six items:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
O₂ content of dry flue gas;
CO content of dry flue gas.

14. The online monitoring method for a fossil fuel converter apparatus according to claim 12, characterized in that, regarding the middle-storage pulverized coal boiler, said restricting conditions formed by measuring boiler operating data select the following six items:
The amount of boiler heat absorption calculated according to the measured boiler steam/water parameters;
Total moisture M of coal calculated according to the coal flow-rate, air flow-rate and temperature of the pulverizing system;
O₂ content of dry flue gas;
CO content of dry flue gas; and
H₂O content of flue gas; and
Amount of dry flue gas.

15. The online monitoring method for a fossil fuel converter apparatus according to claim 1, characterized in that, said fossil fuel is fuel gas.

16. The online monitoring method for a fossil fuel converter apparatus according to claim 1, characterized in that, said fossil fuel is fuel oil.

17. An online monitoring device for a fossil fuel converter apparatus, comprising a computer, a data input passage for collecting converter apparatus operating data, and an output data display/output passage, for monitoring a fossil fuel converter apparatus in real-time, characterized in that, said online monitoring apparatus comprises:
Means for storing reactant compositions, fossil fuel compositions, resultants compositions, and relationship between the fossil fuel compositions and calorific value;
Means for storing an equation set involving the fossil fuel compositions, the reactant compositions and the resultants compositions, according to energy balance relationship and material balance relationship in the combustion process;
Means for providing given conditions for use of the above equation set, wherein said given conditions are equations concerning variable relationship in the above equation set;
Means for, according to measured boiler operating data, assigning the variables in the above equation set to form positive definite conditions of the equation set, wherein the sum of the number of the assigned variables and the number of the above given conditions are equal to the sum of the number of variables of the reactant compositions, number of variables of fossil fuel compositions and number of variables of incomplete resultants compositions; and
Means for finding the solution to the equation set to obtain real-time monitoring data of the fossil fuel converter apparatus.

18. The online monitoring device for a fossil fuel converter apparatus according to claim 17, characterized in that said fossil fuel converter apparatus can be selected from the group consisting of: pulverized coal boiler, circulating fluidized bed boiler, integrated gasification combined circulating power generator, gas-turbine combined circulating power generator, and fuel cells.

19. The online monitoring method for a fossil fuel converter apparatus according to claim 11, characterized in that,
Said forming the restricting conditions for the equation set by measuring boiler operating data so as to realize positive definite conditions of the equation set including:
Selecting six of the following restricting conditions for the equation set formed by measuring boiler operating data:
The amount of boiler heat absorption calculated according to the measured boiler steam/water parameters;
Total moisture M of coal calculated according to the coal flow-rate, air flow-rate and temperature of the pulverizing system;
Total amount of coal feeding;
Total air input;
Amount of dry flue gas;
Amount of fly-ash and slag or ash content of coal;
Carbon content of fly-ash;
O₂ content of dry flue gas;
CO content of dry flue gas; and
H₂O content of flue gas.
